(19) United States
(12) Patent Application Publication
(10) Pub. No.: US 2002/0081733 A1
(43) Pub. Date: Jun. 27, 2002

(54) METHOD TO PREPARE DRUG-RESISTANT, NON-MALIGNANT HEMATOPOIETIC CELLS

(76) Inventors: CATHERINE M. VERFAILLIE, ST. PAUL, MN (US); R. SCOTT MCIVOR, ST. LOUIS PARK, MN (US); ROBERT C. ZHAO, MINNEAPOLS, MN (US)

Correspondence Address: SCHWEGMAN LUNDBERG WOESSNER & KLUTH, PO BOX 2938, MINNEAPOLIS, MN, 55402

(21) Appl. No.: 09/068,817

(22) PCT Filed: Nov. 13, 1996
(86) PCT No.: PCT/US96/18273

(51) Int. Cl. C12N 15/09; C12N 5/00; C12Q 1/68; C07H 21/02; C07H 21/04; C12N 15/85

(52) U.S. Cl. 435/455; 435/6; 514/44; 435/91.1; 435/375; 536/23.1; 536/24.5; 530/387.1

(57) ABSTRACT

A method to prepare drug-resistant, non-malignant hematopoietic cells is provided.

### BACKGROUND OF THE INVENTION

Chronic myelogenous leukemia (CML) is a malignant disease of hematopoietic stem cells (HSC), characterized by the clonal expansion of a transformed, pluripotent HSC containing a Philadelphia chromosome (Ph⁺). At the molecular level, the disease is characterized by a translocation between c-ABL on chromosome 9 and BCR (break point cluster region) on chromosome 22. Both the normal c-ABL and BCR genes are important in hematopoiesis. The BCR/ABL translocation results in the production of a protein, P210^{BCR/ABL}. Compared to P160^{ABL}, P210^{BCR/ABL} has increased tyrosine kinase activity, is located in the cytoplasm, and associates with the actin cytoskeleton and a variety of intracellular signaling molecules, all of which have been associated with malignant transformation. In contrast to normal progenitors, CML progenitors adhere poorly to bone marrow stroma, even though CML progenitors have normal numbers of β1-integrins and other adhesion receptor molecules. The presence of P210^{BCR/ABL} is required and causes the malignant transformation of hematopoietic cells.

Conventional therapy with hydroxyurea, or busulfan, can control CML but does not prevent the inevitable onset of blast crisis, or prolong survival times for the majority of patients. The only curative treatment for CML is the transplantation of HSC from a related, unrelated but HLA-matched, or closely HLA-matched donor. However, this procedure results in significant mortality and morbidity resulting from immunologic disparity between donor and recipient. Moreover, up to 60% of CML patients are ineligible for allogeneic-bone marrow transplantation (BMT) because a suitable donor cannot be located or because of the recipient's age.

An alternative to allogeneic transplantation is autologous bone marrow (BM) or peripheral blood (PB) transplantation after cytoreduction with a combination of high dose chemotherapy or chemoradiotherapy. This type of therapy provides prolonged survival and is associated with a 65% primary engraftment rate, low mortality, and prompt return to normal activities even in older recipients.

Although restoration of Ph⁻ hematopoiesis has been observed in 30-90% of CML patients undergoing transplants, most patients suffer a cytogenetic and/or hematological relapse within one year after transplant. This is a result of contamination of autologous donor cells with Ph⁺ cells and/or residual disease persisting in the host, following the intensive preparative regimen. If the relapse is the result of contaminating donor leukemic cells, then harsher in vitro purging techniques can be employed prior to implantation. However, such techniques can also damage the healthy marrow cells, thereby preventing or delaying reconstitution in some cases. If the relapse arises from residual malignant cells in the patient, then more strenuous ablation would be appropriate. However, ablative techniques are quite toxic.

Although improvements in surgery, radiation therapy, and/or chemotherapy are continuing, intensive efforts are being made to develop new modes of cancer treatment, such as gene therapy. Gene therapy is a technique in which a foreign gene is inserted into donor cells either to correct a genetic error or to introduce a new function to the cells. Several techniques exist for introducing genes into human cells, including the use of calcium phosphate, polycations, lipid vesicles, electric current, microprojectile bombardment, or direct microinjection. The efficiency of gene transfer using these techniques is generally less than 0.01%. Because of the need for high-efficiency transfer of DNA into cells for clinical applications, attention has increasingly turned to the use of viruses, particularly transforming DNA viruses such as papovaviruses, adenoviruses, or retroviruses, as gene delivery systems. These viral vectors have the advantage of infecting multiple cell types with efficiencies up to 100%.

Thus, a continuing need exists for an improved method to produce, select for, and promote the proliferation of non-malignant transplanted hematopoietic cells while inhibiting or preventing the proliferation of residual diseased or malignant transplanted host cells. Such a method could effectively extend hematopoietic remission.

### SUMMARY OF THE INVENTION

The present invention provides a method to eliminate residual neoplastic disease from a host wherein the disease is characterized by the presence of an immature hematopoietic progenitor cell having a well-defined gene rearrangement. The gene rearrangement encodes a mRNA and/or protein, the expression of which promotes and/or enhances the disease. Exemplary diseases which can be treated by the method of the invention include CML, which is associated with a BCR/ABL gene rearrangement (P210), acute lymphoblastic leukemia (ALL), which is associated with a BCR/ABL gene rearrangement (P190), and acute promyelocytic leukemia (APL), which is associated with a PML/RAR gene rearrangement.

The invention provides a method for rendering a host mammal resistant to a cytotoxic agent. The method comprises the introduction of a population of transduced hematopoietic cells into a mammalian host, wherein the transduced cells comprise a preselected DNA molecule comprising (i) a first DNA segment which imparts resistance of a host cell to a cytotoxic agent operably linked to a first promoter functional in the transduced cell, and (ii) a second DNA segment operably linked to a second promoter functional in the transduced cell, wherein the second DNA segment encodes a RNA molecule or polypeptide, the expression of which decreases or inhibits the expression of a RNA molecule or polypeptide which is present in a malignant hematopoietic cell and which is not present in a corresponding non-malignant cell. The RNA molecule which is present in the malignant cell encodes a growth promoting gene product. As used herein, “growth promoting gene product” means a RNA and/or polypeptide, the expression of which confers a growth advantage on a cell relative to a cell which does not express, or has a lower level of expression, of the gene product. Preferably, the growth promoting gene product, e.g., P210^{BCR/ABL}, is encoded by a region of the genome which includes a translocation. The transduced cells are maintained in the host and the host exhibits resistance to the cytotoxic agent. It is preferred that the transduced cells are bone marrow-derived cells from the host. After the transduced cells are introduced into the host, the host is administered the cytotoxic agent.

The expression of the first DNA segment in the transplanted cells confers resistance to the cytotoxic agent to both normal and malignant transduced hematopoietic stem cells. A preferred first DNA segment of the invention can encode resistance to methotrexate, vinblastine, cisplatin, alkylating agents, taxol or anthracyclines, their analogs or derivatives, and the like. The expression of the second DNA segment in the transplanted cells decreases the expression of the RNA molecule or polypeptide which is present in malignant cells, but not present in non-malignant cells, which results in the elimination or inhibition of the malignant phenotype, e.g., adhesion-independent proliferation, IL-3 independent proliferation, inability or decreased ability to adhere to stroma or fibronectin, or tumorigenicity in syngeneic mice. Preferably, the second DNA segment comprises an antisense oligonucleotide (ASO), or encodes a ribozyme or a portion of an antibody. As used herein, the term “antisense oligonucleotide” means a short sequence of nucleic acid which is the reverse complement of at least a portion of a RNA molecule encoded by a gene. The duplex formed by the ASO and the RNA inhibits translation of the RNA, as well as promotes RNA degradation. A preferred method of the invention is the elimination of Ph⁺ stem cells from transplanted bone marrow in CML patients.

The invention also provides an expression cassette comprising a first nucleic acid molecule encoding resistance to a cytotoxic agent operably linked to a first promoter functional in a host cell, such as a mammalian cell, and a second nucleic acid molecule encoding a RNA molecule or polypeptide, the expression of which decreases the expression of a RNA molecule or polypeptide that is present in a malignant cell but not present in a corresponding non-malignant cell. A preferred cassette comprises a second nucleic acid molecule that encodes a RNA which is complementary to a RNA molecule or sequence that encodes a growth promoting gene product.

Another embodiment of the invention is a method of preparing a cytotoxic drug-resistant, non-malignant cell. The method comprises the introduction into a cell of a preselected DNA molecule comprising (i) a first DNA segment comprising a cytotoxic drug resistance gene operably linked to a first promoter functional in the cell, and (ii) a second DNA segment operably linked to a second promoter functional in the cell to provide a transduced cell. The expression of the second DNA segment, which encodes a RNA molecule or polypeptide, decreases the expression of a RNA molecule or polypeptide which is present in a malignant cell and which is not present in a corresponding non-malignant cell. The preselected DNA molecule is then expressed in the transduced cell in the presence of the drug.

As used herein, the term “hematopoietic stem cells (HSC)” means a population of primitive progenitor cells which can provide long term reconstitution of both myeloid and lymphoid cell lineages in a lethally irradiated host when introduced thereinto. An in vitro assay to assess the identity of HSC is not yet available, however, the absolute number of long term culture initiating cells (LTC-IC) correlates with the absolute number of HSC.

As used herein, the term “long term culture initiating cell (LTC-IC)” means a cell that can initiate and sustain long term bone marrow cultures in vitro. Moreover, a LTC-IC can differentiate into myeloid, B-lymphoid, natural killer cell, and T-cell lineages, when LTC-IC are induced to differentiate in vitro by chemical or physical methods or in vivo by transplant into xenogeneic recipients.

As used herein, the term “resistant cell” means a cell which has been genetically modified so that the cell proliferates in the presence of an amount of a drug or cytotoxic agent that inhibits or prevents proliferation of a cell without the modification.

As used herein with respect to a nucleic acid molecule that encodes resistance to an agent or drug, the term “agent or drug resistance” means that the expression of the nucleic acid molecule in a cell permits that cell to proliferate in the presence of the agent or drug to which the gene confers resistance, to a greater extent than the cell can proliferate without the nucleic acid molecule.

As used herein with respect to an agent or drug, the term “therapeutically effective amount” means an amount of an agent or drug that inhibits or prevents proliferation of a non-genetically modified, i.e., by recombinant means, cell in a mammalian host.

### BRIEF DESCRIPTION OF THE FIGURES

FIG. 1. Sequence of antisense oligonucleotides and RT-PCR primers. The nucleotide sequence of b3a2 (SEQ ID NO: 1), b2a2 (SEQ ID NO:2), missense b3a2 (SEQ ID NO:3), missense b2a2 (SEQ ID NO:4), BCR 5′ primer (SEQ ID NO:5), ABL 3′ primer (SEQ ID NO:6), β-actin 5′ primer (SEQ ID NO:7), β-actin 3′ primer (SEQ ID NO:8), BCR exon I 5′ primer (SEQ ID NO:9), BCR exon II 3′ primer (SEQ ID NO: 10), ABL exon Ia 5′ primer (SEQ ID NO: 11), and ABL exon II 3′ primer (SEQ ID NO: 12) is shown.

FIG. 2. Drug sensitivity of untransduced cells. A) NL and CML 34⁺DR⁺ cells were plated in a serum-free methylcellulose assay with increasing MTX concentrations to determine the MTX sensitivity of CFC. B) NL 34⁺DR⁻ cells (LTC-IC) and CML 34⁺DR⁺ (Ph⁺ LTC-IC) cells were incubated for 1 week in serum-free medium and cytokines with increasing MTX concentrations and then replated in LTC for 5 weeks. The number of MTX resistant LTC-IC was determined by replating LTC derived progeny in methylcellulose without MIX. C) NL and CML 34⁺DR⁺ cells were plated directly in methylcellulose with increasing concentrations of taxol. D) NL 34⁺DR⁻ cells (LTC-IC) and CML 34⁺DR⁺ (Ph⁺LTC-IC) were plated in liquid culture for 7 days with increasing concentrations of taxol and then replated in LTC.

FIG. 3. MTX resistance of TYR22-DHFR transduced NL CFC. NL 34⁺DR⁺ cells were transduced with a retroviral vector containing the TYR22-DHFR MTX-resistance gene (LBD, see FIG. 4).

FIGS. 4. LasBD vector. A) Construction of LasBD vector. B) Synthesis of the two copy antisense oligonucleotide sequence. C) Schematic diagram of LasBD vector. Large open boxes represent retroviral long terminal repeat elements. The small shaded box represents the β-actin promoter. The large box marked DHFR represents the TYR22-DHFR gene. The small blackened box represents one copy of an antisense oligonucleotide (ASO) sequence. Arrows represent the direction of transcription.

FIG. 5. ASOs restore CFC adhesion (A) and adhesion-dependent proliferation (B).

FIG. 6. Relative levels of BCR/ABL mRNA in ASO-treated cells.

FIG. 7. Expression of antisense oligonucleotides in transduced cells results in downregulation of BCR/ABL RNA and protein.

FIG. 8. Expression of antisense oligonucleotides in transduced cells normalizes adhesion receptor expression.

FIG. 9. Methotrexate sensitivity of transduced cells.

FIG. 10. BCR/ABL RNA in transduced Ph+ CML cells. LBDBas is a retroviral vector where the TYR22-DHFR gene is linked to a β-actin promoter and the TYR22-DHFR gene is 5′ to a two copy BCR/ABL ASO which is linked to a β-actin promoter.

FIG. 11. Survival of C3H mice after injection of transduced 32D^{P210} cells.

FIG. 12. Expression of antisense oligonucleotides in transduced cells does not inhibit expression of p190^{BCR/ABL}.

FIG. 13. Expression of antisense oligonucleotides in A) 32D^{P910} cells, B) 32D^{P210} cells, and C) MO7e^{P210} cells.

FIG. 14. Expression of antisense oligonucleotides in transduced cells does not affect in vivo survival of 32DP^{p190} cells.

FIG. 15. Expression of c-ABL, c-myc, p53, bcl-2, MAX and BAX in LasBD transduced cells.

### DETAILED DESCRIPTION OF THE INVENTION

While one treatment possibility for CML patients is continued treatment with cytotoxic agents post auto-grafting, the chemotherapeutic agents can affect both Ph⁺ and normal HSC. To avoid damaging the infused donor HSC during chemotherapy, a gene which renders cells resistant to the cytotoxic agent, is transferred into donor HSC prior to grafting. Thus, the transduced HSC, but not the residual diseased host cells, will survive post-transplant chemotherapy. For example, a mutant dihydrofolate reductase (DHFR) gene which renders donor cells significantly more resistant to methotrexate (MTX) than the wild-type DHFR gene, or a multi-drug resistance (MDR) gene, can be employed in the practice of the invention. Since CML HSCs are known to be at least as sensitive to MTX as their normal counterparts (see below), transduction of donor BM with a DHFR gene, such as TYR²²-DHFR, allows transduced HSCs to survive MTX administration post-transplant.

Because complete and lasting reconstitution of the recipient marrow requires that a population of HSC be introduced into the recipient, methods are employed to select CD34⁺HLA-DR⁻ cells from early chronic phzse CML BM which are highly enriched in Ph⁺ progenitors capable of initiating long term bone marrow cultures (LTC-IC). Ph⁺ LTC-IC are present in the CML CD34⁺HLA-DR⁺ BM population and can also be found in the DR⁻ BM fraction of patients with late chronic phase CML or with accelerated phase disease.

To select against malignant donor Ph⁺ progenitors, donor cells are exposed to a BCR/ABL antisense oligonucleotide (ASO). BCR/AB L ASOs have been shown to inhibit cell proliferation and apoptosis in CML cell lines, to inhibit colony formation in blast crisis CML and, possibly, chronic phase CML progenitors, to restore the ability of CML progenitors to adhere to stroma, and to inhibit the unregulated proliferation of CML progenitors. Thus, while not eliminating the malignant phenotype, the presence of BCR/ABL ASOs in CML progenitor cells results in increased adhesion and decreased proliferation of CML progenitors, preventing the uncontrolled expansion of these progenitors.

However, the usefulness of ASO therapy for CML auto-grafting is lessened by the fact that oligonucleotides are unstable in the extracellular and intracellular environment, and poorly internalized. This problem can be overcome by introducing a BCR/ABL ASO into a retroviral vector. The introduction of a BCR/ABL ASO in such a vector permits the stable introduction of these ASOs into the cell nucleus so that the sequences can be constitutively expressed. These vectors can then be tested in vitro in cell lines that have a BCR/ABL gene rearrangement for inhibition of BCR/ABL mRNA and protein, e.g., P210^{BCR/ABL}, expression.

Vectors containing both a cytotoxic agent resistance gene, for example DHFR, and a breakpoint specific ASO (e.g., BCR/ABL b3a2), a gene encoding a ribozyme specific for the breakpoint in a gene rearrangement, or a gene encoding a portion of an antibody which specifically binds to the protein encoded by a gene rearrangement, can be introduced into a cell line having a BCR/ABL gene rearrangement to determine whether an agent resistant phenotype can be selected in cells with decreased BCR/ABL mRNA and P210^{BCR/ABL} expression. The vectors can then be introduced into freshly isolated normal and Ph⁺ progenitors to determine what effect the expression of the breakpoint specific ASO, the gene encoding a ribozyme specific for the breakpoint in a gene rearrangement, or the gene encoding a portion of an antibody which specifically binds to the protein encoded by a gene rearrangement, has on these cells, and whether the introduction of a cytotoxic agent-resistance gene can confer resistance to the cytotoxic agent.

Transduced cells can also be introduced into chimeric transplant models and animal models of CML. Murine hosts suitable for these types of studies include SCID mice, NOD-SCID mice, or BNX mice. The addition of human hematopoietic growth factors and/or human stromal cells to these animals may be necessary to permit the growth and maintenance of the transplanted cells.

### A. Hematopoietic Stem Cells

The human hematopoietic system is populated by cells of several different lineages. These “blood cells” may appear in bone marrow, the thymus, lymphatic tissue(s) and in peripheral blood. Within any specific lineage, there are a number of maturational stages. In most instances, the more immature developmental stages occur within bone marrow while the more mature and final stages of development occur in peripheral blood.

There are two major lineages: The myeloid lineage which matures into red blood cells, granulocytes, monocytes and megakaryocytes; and the lymphoid lineage which matures into B lymphocytes and T lymphocytes. Within each lineage and between each lineage, antigens are expressed differentially on the surface and in the cytoplasm of the cells in a given lineage. The expression of one or more antigens and/or the intensity of expression can be used to distinguish between maturational stages within a lineage and between lineages.

Assignment of cell to lineage and to a maturational stage within a cell lineage indicates lineage commitment. There are cells, however, which are uncommitted to any lineage (i.e., “progenitor” cells) and which, therefore, retain the ability to differentiate into each lineage. These undifferentiated, pluripotent progenitor cells are referred to as the “hematopoietic stem cells (HSCs).”

All of mammalian hematopoietic cells can, in theory, be derived from a single stem cell. In vivo, the stem cell is able to self-renew, so as to maintain a continuous source of pluripotent cells. In addition, when subject to particular environments and/or factors, the stem cells may differentiate to yield dedicated progenitor cells, which in turn may serve as the ancestor cells to a limited number of blood cell types. These ancestor cells will go through a number of stages before ultimately yielding mature cells.

The benefit of identifying and obtaining a pure population of stem cells is most readily recognized in the field of gene therapy. Gene therapy seeks to replace or repopulate the cells of the hematopoietic system which contain a defective gene with cells that do not contain the defective gene but instead contain a “normal” gene. Thus, using conventional recombinant DNA techniques, a “normal” gene is isolated, placed into a viral vector, and the viral vector is transfected into a cell capable of expressing the product coded for by the gene. The cell then must be introduced into the patient. If the “normal” gene product is produced, the patient is “cured” of the condition.

However, the transformed cells must be capable of continual regeneration as well as growth and differentiation. Thus, while Kwok et al. (PNAS USA, 83, 4552 (1986)) demonstrated that gene therapy was possible using retroviral vector-transduced progenitor cells in dogs, the transduced cells were not capable of self-renewal. Thus, the “cure” was only temporary.

Other difficulties encountered in stem cell gene therapy is that the stem cell population constitutes only a small percentage of the total number of leukocytes in bone marrow. Weissman et al. (EPO 341,966) reported that murine bone marrow contains only about 0.02-0.1% pluripotent stem cells. Moreover, the introduction of between 20-30 of these stem cells per recipient are necessary to rescue 50% of a group of lethally-irradiated mice. See Weissman et al., supra and Spangrude et al., Science, 241, 58 (1988).

The development of cell culture media and conditions that will maintain stem cells in vitro for the extended periods of time required for the procedures involved in gene therapy, identification of growth factors, thorough characterization of cell morphologies and the like, has presented a unique set of obstacles. To date, successful in vitro stem cell cultures have depended on the ability of the laboratory worker to mimic the conditions which are believed to be responsible for maintaining stem cells in vivo.

For example, hematopoiesis occurs within highly dense cellular niches within the bone marrow in the adult, and in similar niches within the fetal yolk sac and liver. Within these niches, stem cell differentiation is regulated, in part, through interactions with local mesenchymal cells or stromal cells. Mammalian hematopoiesis has been studied in vitro through the use of various long-term marrow culture systems. T. M. Dexter et al., in J. Cell Physiol., 91, 335 (1977) described a murine system from which spleen colony-forming units (CFU-S) and granulocyte/macrophage colony forming units (CFU-GM) could be detected for several months, with erythroid and megakaryocytic precursors appearing for a more limited time. Maintenance of these cultures was dependent on the formation of an adherent stromal cell layer composed of endothelial cells, adipocytes, reticular cells, and macrophages.

These methods were soon adapted for the study of human bone marrow. Human long-term culture system were reported to generate assayable hematopoietic progenitor cells for 8 or 9 weeks, and, later, for up to 20 weeks (See, S. Gartner, et al., PNAS USA, 77, 4756 (1980); F. T. Slovick et al., Exp. Hematol., 12, 327 (1984). Such cultures were also reliant on the preestablishment of a stromal cell layer which must frequently be reinoculated with large, heterogeneous populations of marrow cells. Hematopoietic stem cells have been shown to home and adhere to this adherent cell multilayer before generating and releasing more committed progenitor cells (M. Y. Gordon et al., J. Cell Physiol., 130, 150 (1987)).

Stromal cells are believed to provide not only a physical matrix on which stem cells reside, but also to produce membrane-contact signals and/or hematopoietic growth factors necessary for stem cell proliferation and differentiation. This heterogenous mixture of cells comprising the adherent cell layer presents an inherently complex system from which the isolation of discrete variables affecting stem cell growth has proven difficult. Furthermore, growth of stem cells on a stromal layer makes it difficult to recover the hematopoietic cells or their progeny efficiently.

Stem cells can also be cultured effectively in vitro, in stromal feeder cell-conditioned medium, with or without added cytokines, as taught in U.S. Pat. Nos. 5,436,151 and 5,460,964.

### B. Expression Cassettes

The recombinant or preselected DNA sequence or segment, used to prepare expression cassettes for transformation, may be circular or linear, double-stranded or single-stranded. Generally, the preselected DNA sequence or segment is in the form of chimeric DNA, such as plasmid DNA, that can also contain coding regions flanked by control sequences which promote the expression of the preselected DNA present in the resultant cell line. As used herein, “chimeric” means that a vector comprises DNA from at least two different species, or comprises DNA from the same species, which is linked or associated in a manner which does not occur in the “native” or wild type of the species.

Aside from preselected DNA sequences that serve as transcription units for drug resistance, antisense oligonucleotides, or portions thereof, a portion of the preselected DNA may be untranscribed, serving a regulatory or a structural function. For example, the preselected DNA may itself comprise a promoter that is active in mammalian cells, or may utilize a promoter already present in the genome that is the transformation target. Such promoters include the β-actin promoter, the CMV promoter, as well as the SV40 late promoter and retroviral LTRs (long terminal repeat elements), although many other promoter elements well known to the art may be employed in the practice of the invention. A preferred promoter useful in the practice of the invention is the β-actin promoter. Another preferred promoter useful in the practice of the invention is a retroviral LTR promoter. Yet another preferred promoter is a polIII promoter.

Other elements functional in the host cells, such as introns, enhancers, polyadenylation sequences and the like, may also be a part of the preselected DNA. Such elements may or may not be necessary for the function of the DNA, but may provide improved expression of the DNA by affecting transcription, stability of the mRNA, or the like. Such elements may be included in the DNA as desired to obtain the optimal performance of the transforming DNA in the cell.

“Control sequences” is defined to mean DNA sequences necessary for the expression of an operably linked coding sequence in a particular host organism. The control sequences that are suitable for prokaryotic cells, for example, include a promoter, and optionally an operator sequence, and a ribosome binding site. Eukaryotic cells are known to utilize promoters, polyadenylation signals, and enhancers.

“Operably linked” is defined to mean that the nucleic acids are placed in a functional relationship with another nucleic acid sequence. For example, DNA for a presequence or secretory leader is operably linked to DNA for a polypeptide if it is expressed as a prepolypeptide that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accord with conventional practice.

The preselected DNA to be introduced into the cells further will generally contain either a selectable marker gene or a reporter gene or both to facilitate identification and selection of transformed cells from the population of cells sought to be transformed. Alternatively, the selectable marker may be carried on a separate piece of DNA and used in a co-transfonnation procedure. Both selectable markers and reporter genes may be flanked with appropriate regulatory sequences to enable expression in the host cells. Useful selectable markers are well known in the art and include, for example, antibiotic, cytotoxic agent and herbicide-resistance genes, such as neo, hpt, dhfr, mdr, bar, aroA and the like. See also, the genes listed on Table 1 of Lundquist et al. (U.S. Pat. No. 5,848,956).

Selection of transduced cells can also be accomplished by transducing cells with a gene that encodes a cell surface protein, e.g., nerve growth factor receptor. Cells which express the transduced receptor can then be identified, e.g., by FACS analysis or by passing the cells over a column to which the receptor-specific ligand is covalently coupled.

Reporter genes are used for identifying potentially transformed cells and for evaluating the flnctionality of regulatory sequences. Reporter genes which encode for easily assayable polypeptides are well known in the art. In general, a reporter gene is a gene which is not present in or expressed by the recipient organism or tissue and which encodes a polypeptide whose expression is manifested by some easily detectable property, e.g., enzymatic activity. Preferred genes include the chloramphenicol acetyl transferase gene (cat) from Tn9 of E. coli, the beta-glucuronidase gene (gus) of the uidA locus of E. coli, and the luciferase gene from firefly Photinus pyralis. Expression of the reporter gene is assayed at a suitable time after the DNA has been introduced into the recipient cells.

The general methods for constructing recombinant DNA which can transform target cells are well known to those skilled in the art, and the same compositions and methods of construction may be utilized to produce the DNA usefuil herein. For example, J. Sambrook et al., Molecular Cloning: A Laboratory Manual, Cold Spring Harbor Laboratory Press (2d ed., 1989), provides suitable methods of construction.

It is preferred that the expression cassettes of the invention are retroviral expression cassettes, i.e., the RNA transcribed from the expression cassettes can be packaged into virions and the RNA transmitted to another cell. Preferably, the copy number of ASOs present in the expression cassettes of the invention is at least about 2-10 copies, more preferably at least about 3-8 copies, and more preferably at least about 4-6 copies. The length of the ASO sequence is preferably at least about 12-50 nucleotides, more preferably at least about 14-40, and more preferably at least about 15-30, nucleotides in length. It is preferred that the ASO sequence is linked to a polIII promoter.

Another preferred expression cassette comprises a nucleic acid sequence which encodes a ribozyme, e.g., a hammerhead ribozyme which is specific for the b3a2 BCR/ABL RNA. Ribozymes are small RNA molecules capable of catalyzing RNA cleavage reactions in a sequence specific manner. Preferably, the nucleic acid molecule encoding the ribozyme, which comprises the conserved hammerhead sequences flanked by 3′ and 5′ sequences which are complementary to the breakpoint in a gene rearrangement, is linked to a polIII promoter.

Yet another preferred expression cassette comprises a nucleic acid sequence encoding a single chain Fv (sFv) of an antibody (“intrabody”) which is specific for a protein that is expressed in a malignant cell but not in a non-malignant cell. For example, the intrabody can specifically bind P210^{BCR/ABL}, e.g., the intrabody can comprise a portion of the antibody secreted by the hybridoma 8E9 line. The expression of the intrabody may divert the P210^{BCR/ABL} protein from its sub-membrane cytoskeletal location, or may inactivate the activity of P210^{BCR/ABL}, and thus inhibit the function of the P210^{BCR/ABL} protein. The sFv region can comprise the V_{L} and V_{H} domains which are covalently linked by a polypeptide linker region. The sFv can also be coupled to sequences that direct the intrabody to a specific intracellular location, e.g., the endoplasmic reticulum.

### C. Mammalian Gene Transfer

Gene transfer methods used in mammalian cells can be classified as physical or biological processes. Physical methods include DNA transfection, lipofection, particle bombardment, microinjection and electroporation. Biological methods include the use of DNA and RNA viral vectors. The main advantage of physical methods is that they are not associated with pathological or oncogenic processes of viruses. However, they are less precise, often resulting in multiple copy insertions, random integration, disruption of foreign and endogenous gene sequences, and unpredictable expression. For human gene therapy, it is desirable to use an efficient means of precisely inserting a single copy gene into the host genome. Viral vectors, and especially retroviral vectors, have become the most widely used method for inserting genes into human cells. Other viral vectors can be derived from poxviruses, herpes simplex virus I, adenoviruses and adeno-associated viruses, and the like. However, most of the current and proposed gene therapy clinical protocols employ retroviral vectors.

Retroviruses are single-stranded RNA viruses which replicate viral RNA into DNA by reverse transcription. Upon replication in the host cell, the viral DNA is inserted into the host chromosome, where it becomes a provirus. Due to their efficiency at integrating into host cells, retroviruses are considered to be one of the most promising vectors for human gene therapy. These vectors have a number of properties that lead them to be considered as one of the most promising techniques for genetic therapy of disease. These include: (1) efficient entry of genetic material present in the vector into cells; (2) an efficient process of entry into target cell nucleus; (3) relatively high levels of gene expression; (4) minimal pathological effects on target cells; and (5) the potential to target to particular cellular subtypes through control of the vector-target cell binding and tissue specific control of gene expression.

Retroviral genomes consist of cis-acting and trans-acting gene sequences. The cis regions include the long terminal repeat (LTR) transcriptional promoter and DNA integration sites, the two primer binding sites required for reverse transcription of DNA from viral RNA, and the packaging signals required for efficient packaging of viral RNA into virions. The LTR is found at both ends of the proviral genome. Trans-functions include the proteins encoded by the gag, pol, and env genes, which are located between the LTRs. Gag and pol encode, respectively, internal viral structural and enzymatic proteins. Env encodes the viral glycoprotein which confers infectivity and host range specificity on the virus. A retroviral vector generally consists of cis sequences and the replacement of the trans sequences with a gene(s) of interest. The trans functions can be provided by expression the trans sequences in a helper cell or by a helper virus. See U.S. Pat. No. 5,354,674 for a discussion of the use of retrotransposon vectors, which are related to retroviral vectors, in mammalian gene transfer.

While previous results indicated that retroviral infection of HSCs is inefficient, most likely due to the quiescent state of a vast majority of HSCs, recent evidence suggests that up to 50% of human steady state bone marrow derived LTC-IC can be transduced with a retroviral vector when those cells are pre-incubated with stromal conditioned media (SCM⁺) containing IL3 (IL3⁺) and MIP-1α, and when LTC-IC are cocultured with stromal feeders, FN, or immobilized β1-integrin antibodies during the transduction period. Moreover, CML LTC-IC have a higher rate of transduction relative to normal LTC-IC, probably due to the higher proliferative capacity of these malignant cells. Thus, culture conditions can be modified to enhance the transduction of HSCs by retroviral vectors.

The invention will be further described by reference to the following detailed examples.

### EXAMPLE 1

### Sensitivity of CML Precursors to Cytotoxic Agents

Although methotrexate (MTX) is not routinely used in the treatment of CML, and can induce hematopoietic and gastrointestinal toxicity, the introduction of a MTX-resistant DHFR gene into HSC can overcome the hematopoeitic and gastrointestinal toxicity observed with MTX administration and allows the administration of higher doses of MTX, which may lead to enhanced tumor elimination.

Likewise, although taxol is not routinely used to treat CML, normal and chronic phase CML precursors express low levels of a multidrug resistance gene (MDR) which encodes a transmembrane protein that pumps naturally occurring toxins, e.g., taxol and colchicine, out of the cell. Thus, chronic phase CML precursors may be particularly susceptible to naturally occurring toxins, such as taxol.

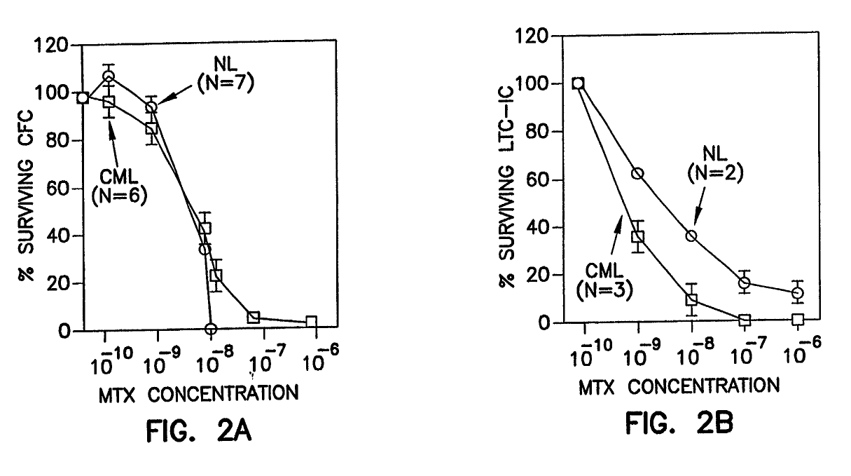

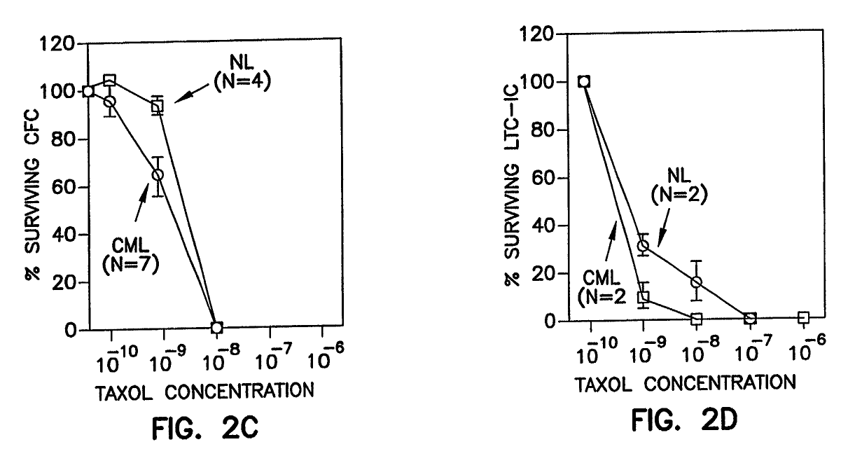

To determine the MTX sensitivity of normal (NL) and CML CFC cells, NL and CML 34⁺DR⁺ cells were plated in a serum-free methylcellulose assay with increasing MTX concentrations (FIG. 2A). To determine the MTX sensitivity of NL and CML LTC-IC cells, NL 34⁺DR⁻ cells (LTC-IC) and CML 34⁺DR⁺ (Ph⁺ LTC-IC) cells were also incubated for I week in serum-free medium and cytokines, with increasing MTX concentrations. The cells were then replated in LTC for 5 weeks. The number of MTX resistant LTC-IC was determined by replating LTC derived progeny in methylcellulose assay without MTX (FIG. 2B). These studies demonstrated that CML 34⁺DR⁺ CFC and LTC-IC are at least equally sensitive to MIX as NL 34⁺DR⁺ CFC and NL 34⁺DR⁻ LTC-IC.

To determine the taxol sensitivity of NL and CML cells, NL and CML 34⁺DR⁺ and 34⁺DR⁻ cells were plated either in liquid culture for 7 days with increasing concentrations of taxol and then replated in LTC (FIG. 2D). NL and CML cells were also plated directly in methylcellulose with increasing concentrations of taxol (FIG. 2C). The results of these studies demonstrated equal sensitivity of CML and NL progenitors to taxol.

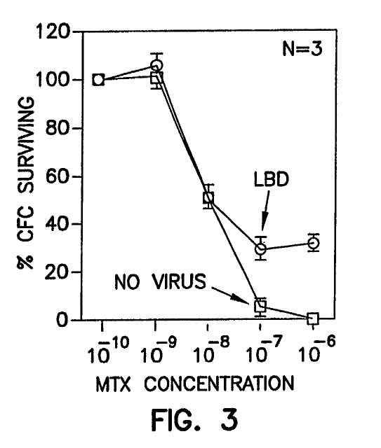

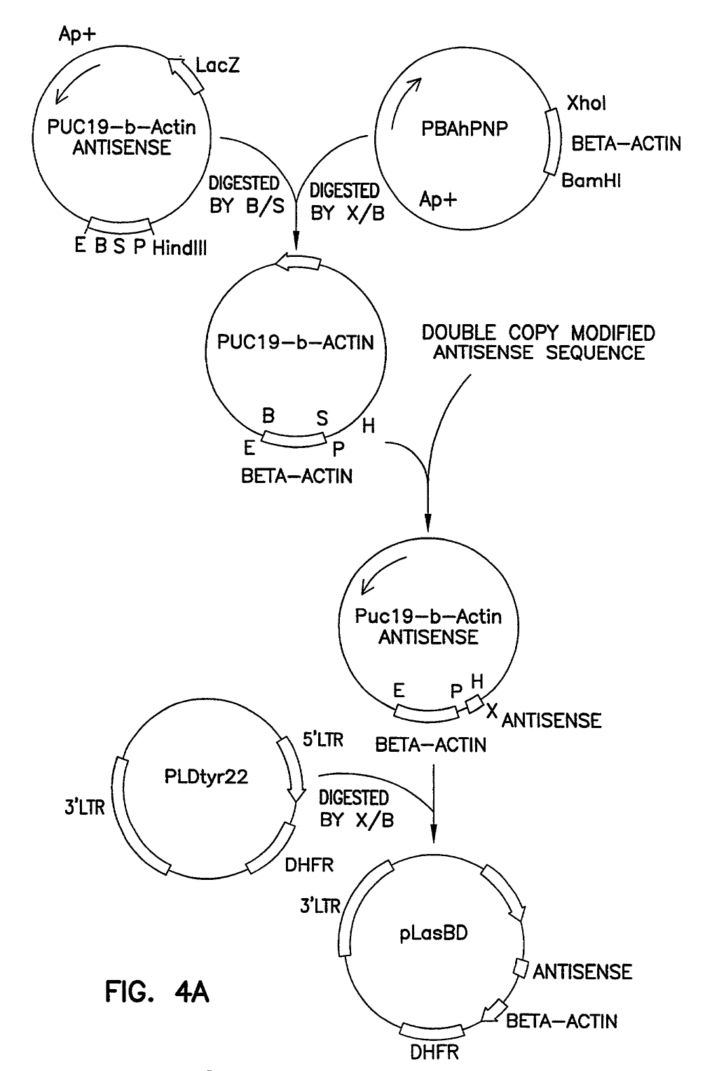

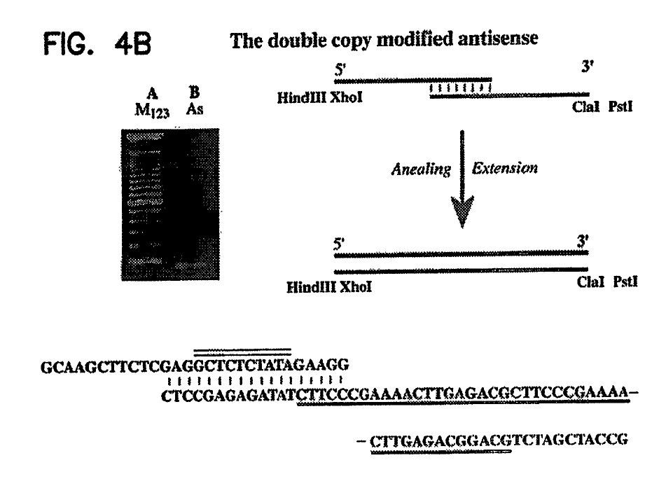

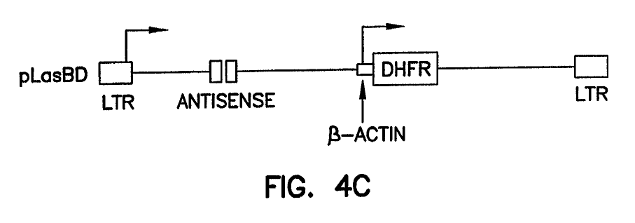

To evaluate the effect of transduction of NL 34⁺DR⁺ cells with a retroviral vector containing the TYR22-DHFR MIX-resistance gene (LBD, see FIG. 4) on the MTX resistance of CFC, NL34⁺DR⁺ cells were infected with LBD. Twenty percent (20%) of LBD transduced NL CFC were resistant to 2 log higher MTX concentrations (10⁻⁶ M) than control CFC that were not transduced (FIG. 3).

Thus, CML precursors are sensitive to both MTX and taxol. Moreover, the introduction of a MTX-resistance gene into NL 34⁺DR⁺ cells renders those cells resistant to MTX.

### EXAMPLE 2

### Antisense Oligonucleotide Expression Can Restore Adhesion and Adhesion-Mediated Growth Regulation in CML

In contrast to NL CFC and LTC-IC, only 20% of Ph+ CML CFC and 40% of Ph+ CML LTC-IC adhere to BM stroma. Furthermore, these progenitors fail to adhere to fibronectin (FN). Moreover, in contrast to NL CFC, CML CFC proliferate continuously, even when in contact with stroma or FN. In addition, the crosslinking of β1-integrin receptors on NL, but not CML, CFC results in the inhibition of progenitor proliferation, although 70-90% of CML progenitors express β1-integrins.

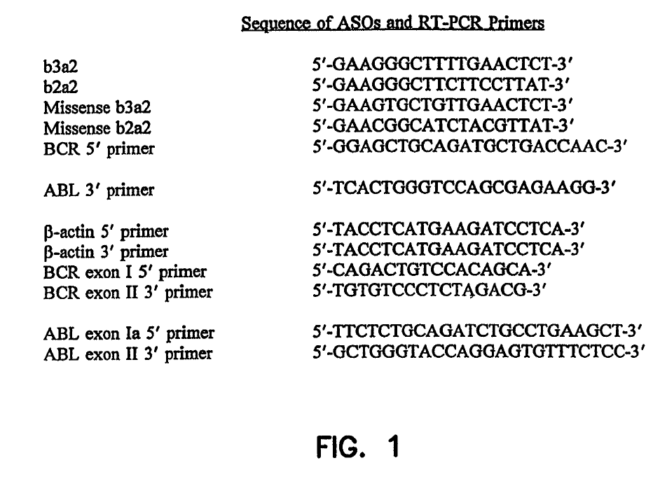

To determine whether inhibition of P210 by ASOs can inhibit and thus restore normal adhesion and adhesion-mediated growth regulation, CML 34⁺ DR⁺ cells were incubated with ASOs. CML 34⁺ DR⁺ cells (n=7, one patient with a b2a2 breakpoint and 6 patients with a b3a2 breakpoint, see FIG. 1) were incubated in serum-free medium supplemented with picogram amounts of G-CSF, IL6, SCF, LIF, MIP-1α with or without 40 μg/ml ASOs for 36 hours prior to adhesion and proliferation assays. Incubation of NL CFC with this growth factor mixture induced proliferation of 40-50% of CFC without affecting their adhesion to, or regulation by, stroma or FN. ASOs were not added further during the methylcellulose assays performed after adhesion and thymidine suicide assays.

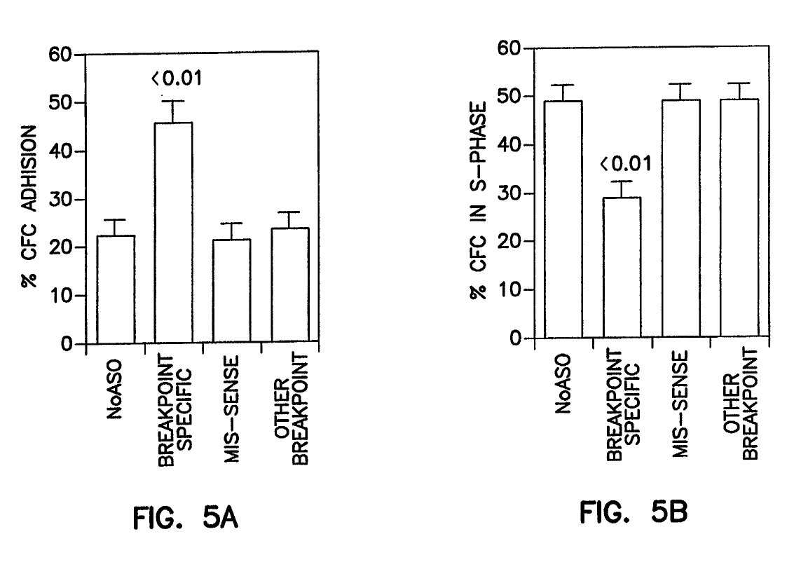

Breakpoint specific ASOs did not change CML colony growth. Breakpoint specific ASOs did, however, restore adhesion to stroma and subsequent proliferation inhibition (FIG. 5). Likewise, breakpoint specific ASOs restored adhesion to FN and restored proliferation inhibition observed following contact with FN or following cross-linking of the α4, α5 or β1 integrin on CML CFC. Moreover, the observed effects were not due to non-specific factors, as NL DR⁺ cells cultured under the same conditions showed no difference in the number of CFC, or in the number of adhering or proliferating CFC, when cultured with or without ASOs.

To further ensure that the observed results in CML cells were due to sequence specific inhibition, missense oligonucleotides were used as well as ASOs against a breakpoint not present in those cells, i.e., that the TAT sequence located 5′ in the b2a2 antisense sequence which may have nonspecific toxic effects independent of the antisense sequence was not responsible for the results. In addition, the breakpoint specific ASOs did not contain the sequences CpG or GpGpGpG which are commonly associated with sequence non-specific metabolic toxicity to cells. Neither ASOs against the other breakpoint nor missense oligonucleotides suppressed CML CFC growth, or effected adhesion to and proliferation inhibition by stroma or FN.

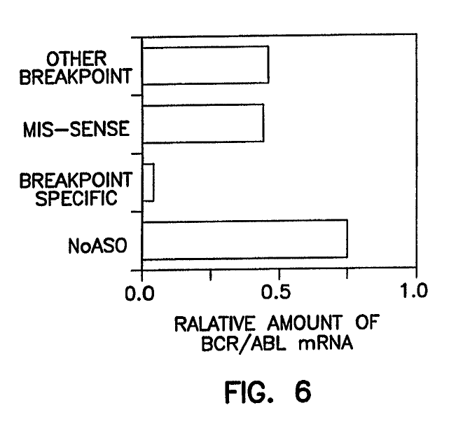

To demonstrate a causal relation between inhibition of p210^{BCR/ABL} by breakpoint specific ASOs and restored adhesion/proliferation inhibition, the expression levels of BCR/ABL mRNA and P₂₁₀^{BCR/ABL} levels in CML 34⁺DR⁺ cells exposed for 36 hours to breakpoint specific ASOs were evaluated by RT-PCR (30 cycles) and Western blot analysis. The relative amount of BCR/ABL mRNA present was calculated as the amount of amplifiable BCR/ABL mRNA divided by the amount of amplifiable β-actin mRNA. Breakpoint specific ASOs suppressed BCR/ABL mRNA (FIG. 6) and protein levels significantly more than missense or ASOs against the other breakpoint.

These studies indicated that although ASOs do not result in death of CML CFC, they restore β1-integrin-dependent adhesion and subsequent transfer of proliferation inhibitory signals in a sequence specific manner. This indicates that elimination of the BCR/ABL message can results in phenotypical normalization of Ph⁺ prog enitors.

### EXAMPLE 3

### Antisense Oligonucleotide Expression in Cells Transduced With BRL/ABL ASO Vectors

To introduce a nucleic acid molecule which comprises both a drug resistance gene and a BCR/ABL ASO into hematopoietic cells, a series of retroviral constructs was developed. An oligonucleotide with two sequential BCR/ABL (b3a2 breakpoint, see FIG. 1; 25 base pairs in length; ASO(2)) ASOs on either side of a short linker region, preferably 10-20 base pairs in length, was synthesized.

Two retroviral-based vectors were constructed. One vector, LasBD, incorporated the TYR22-DHFR gene as well as two 20-mer anti-b3a2 ASO sequences connected by a 10 base pair synthetic linker (FIG. 4). The ASO(2) sequence was cloned unidirectionally upstream from the DHFR gene under the transcriptional regulation of the β-actin promoter. A control retroviral vector, containing TYR²²-DHFR was also constructed (LBD). The sequence of each vector was verified by restriction endonuclease mapping.

Vectors were then shuttle packaged in PA317 producer cells by incubating PA317 cells with LasBD or LBD in the presence of polybrene. Retrovirus containing supernatants, having a titer of approximately 5×10⁶ virions/ml, were then used to transduce MO7e^{B/A} (MO7e^{P210}) and 32D^{B/A} (32D^{P210}) cells. 32D cells, like MO7e cells, are IL3 dependent in vitro. Upon transduction with BCR/ABL cDNA, 32D^{B/A} cells, unlike MO7^{B/A} cells, are tumorigenic in syngeneic C3H mice in vivo. Transduced cells were selected in the presence of 0.25M MTX and IL-3 for 14 days. Cells were also subcloned at 1,000 cells/well, and transduced cells were selected as described above.

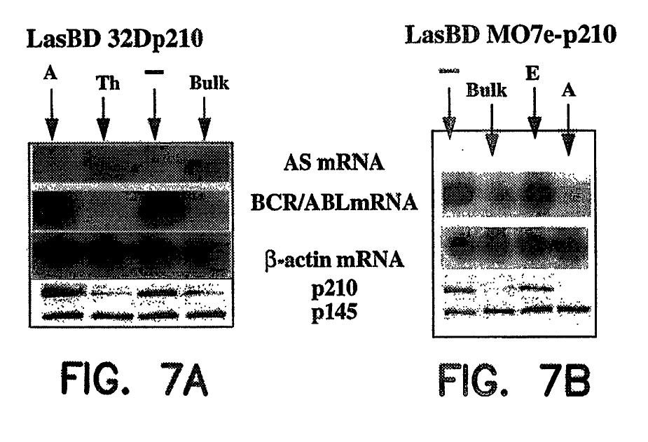

Bulk transduced cells as well as subdlones of the LasBD transduced MO7e^{B/A} and 32D^{B/A} were evaluated. For bulk selected cells, the level of BCR/ABL RNA decreased in both MO7e^{B/A} and 32D^{B/A} transduced cells (FIG. 7). When subclones were examined, some clones showed almost a complete absence of BCR/ABL RNA. Similar results were observed when P210 protein levels were examined. Semi-quantitative reverse-transcriptase (RT-PCR) assays were employed to assess the level of ASO RNA expression in both bulk selected cells and subcloned cells (FIG. 7). The reduction in BCR/ABL RNA and P210 levels was inversely correlated to the levels of ASO RNA expression in bulk, or subcloned, LasBD-transduced cells.

When LasBD transduced-32D^{P210} or MO7e^{P210} cells were cultured in the presence of IL-3, clones in which P210 was eliminated expanded significantly less than 32D^{P210}, MO7e^{P210} or clones that contained some P210. This reduction in P210 expression was correlated with a decrease in c-myc expression, indicating that LasBD can eliminate the activity of the RAS/MAPK/Myc pathway.

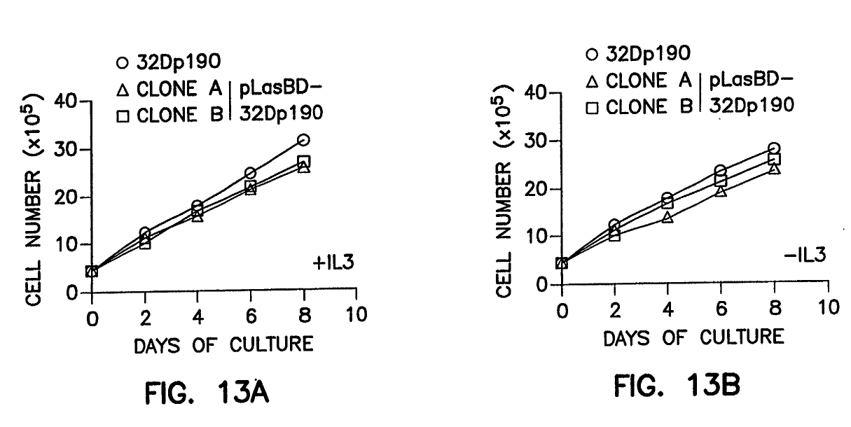

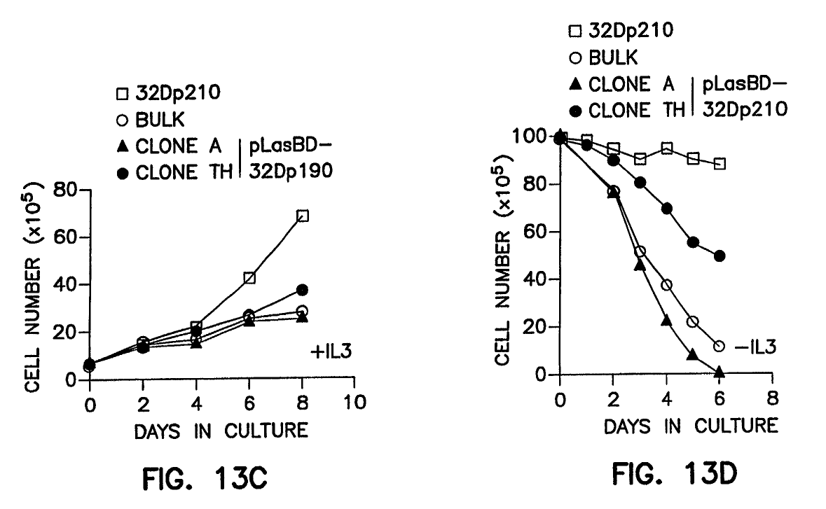

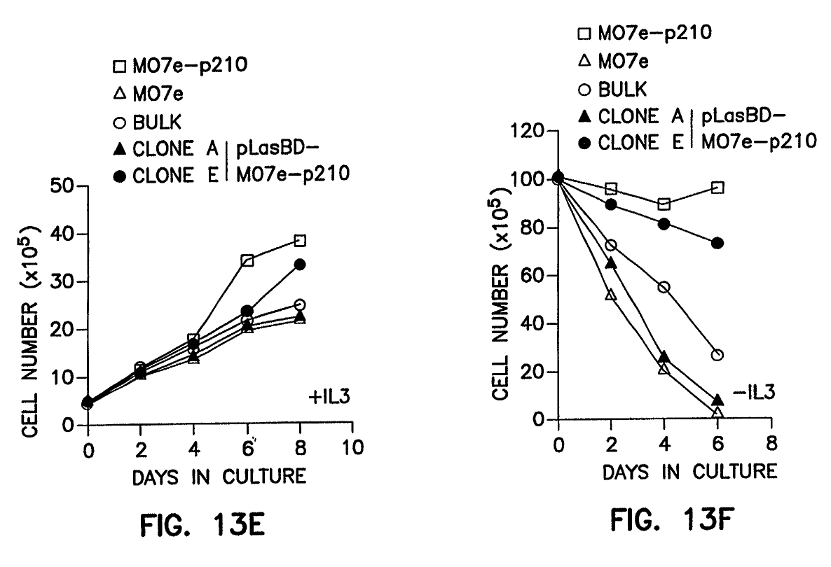

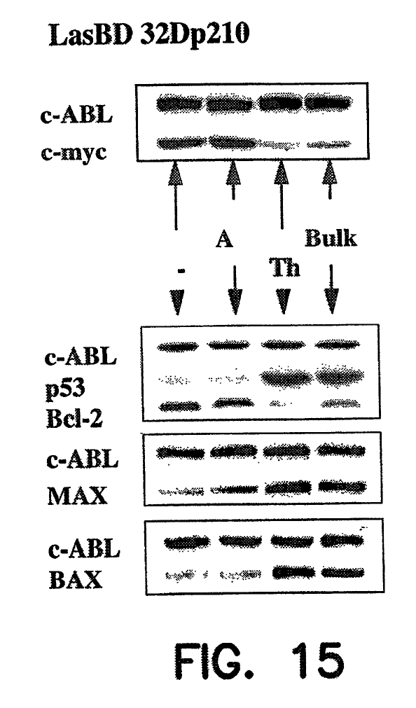

In contrast to MO7e and 32D cells, which apoptose when IL-3 is withdrawn, 32D^{P210} or MO7e^{P210} cells were IL-3 independent, i.e., they continue to grow after IL-3 withdrawal. However, when IL-3 is withdrawn from LasBD transduced-32D^{P210} or MO7e^{P210} cells, cell death was observed 4-6 days after IL-3 was withdrawn, which was more pronounced in clones where P210 was eliminated (clone TH versus clone A) (FIG. 13). As for parent IL3 -dependent MO7e or 32D cells, this was associated with increased p53, max and bax and decreased bcl-2 protein levels (FIG. 15), suggesting that elimination of P210 results in apoptosis in the absence of IL3. FACS analysis using the DNA binding dye, 7AAD, confirmed that LasBD transduced cells apoptosed without IL3.

CML progenitor cells express significantly more α4β1, α5β1 and CD44 receptors than their NL counterparts. However, adhesion of primary Ph+ CML CFC and LTC-IC through β1 integrins is defective, suggesting that abnormal function of integrins may underlie the abnormal premature circulation of CML progenitors in the blood.

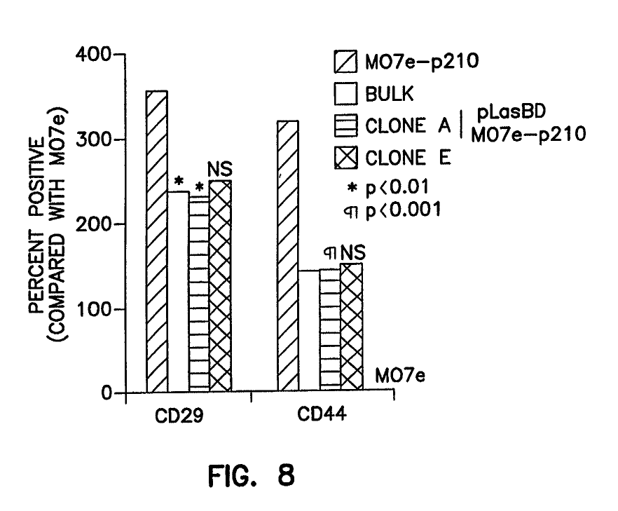

To determine whether LasBD could restore normal adhesive function in CML, the expression of CD44 and β1-integrin on MO7e, MO7e^{P210} and MO7e^{P210} cells transduced with LasBD was examined. Expression of both CD44 and β1-integrin was upregulated in MO7e^{P210} cells compared to MO7e cells (FIG. 8). Expression of both CD44 and β1-integrin was downregulated in LasBD transduced MO7e^{P210} cells compared to MO7e cells. Moreover, incubation of anti-β1-integrin antibodies and CML or NL progenitor cells results in the formation of caps on NL but not on CML progenitor cells. Likewise, incubation of anti-β1-integrin antibodies and MO7e^{P210} or MO7e cells resulted in the formation of caps on MO7e cells but not on MO7e^{P210} cells. Similar studies on LasBD transduced cells resulted in the formation of caps. Thus, LasBD normalizes adhesion receptor expression.

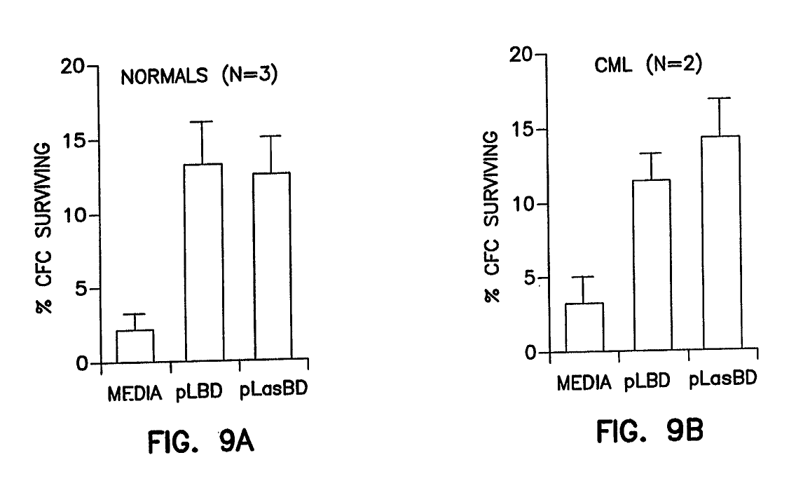

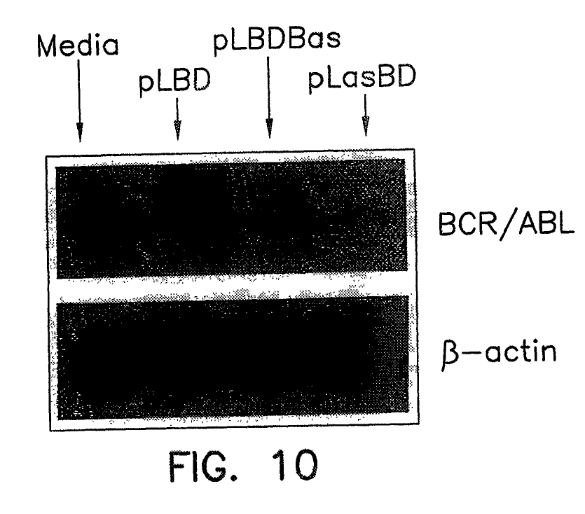

Primary Ph+ CML CD34+HLA−DR+ and NL CD34+HLA−DR+ cells were transduced with the LBD or LBDBas. Cells were cultured in methylcellulose assay in the presence or absence of 5×10⁻⁸ M MTX and colonies enumerated. Transduction with either LBD or LasBD results in similar MTX resistance (FIG. 9). Further, cells recovered after 2 weeks of culture in the presence of MTX were subjected to RT-PCR to detect BCR/ABL mRNA. Transduction of CML DR⁺ cells with LasBD but not LBDBas resulted in nearly complete elimination of the BCRIABL mRNA signal (FIG. 10). These studies demonstrate that the LasBD vector results in high level transcription of the AS sequence in >70% of subclones, leading to elimination of BCR/ABL mRNA and P210^{BCR/ABL} proteins. This results in “normalization” of cell function.

### EXAMPLE 4

### In Vivo Effect of ASO BCR/ABL on Tumorigenicity

To determine if ASO BCR/ABL expression in vivo could inhibit tumorigenicity, BCR/ABL cDNA transduced 32D cells were transplanted into syngeneic mice. 32D cells are a “normal”, non-leukemic cells derived from long-term marrow cultures which are not tumorigenic when transplanted in syngeneic C3H animals and IL3 dependent in vitro. Once transfected with p210^{BCR/ABL} cDNA, the cell line becomes IL3 independent and tumorigenic in vivo.

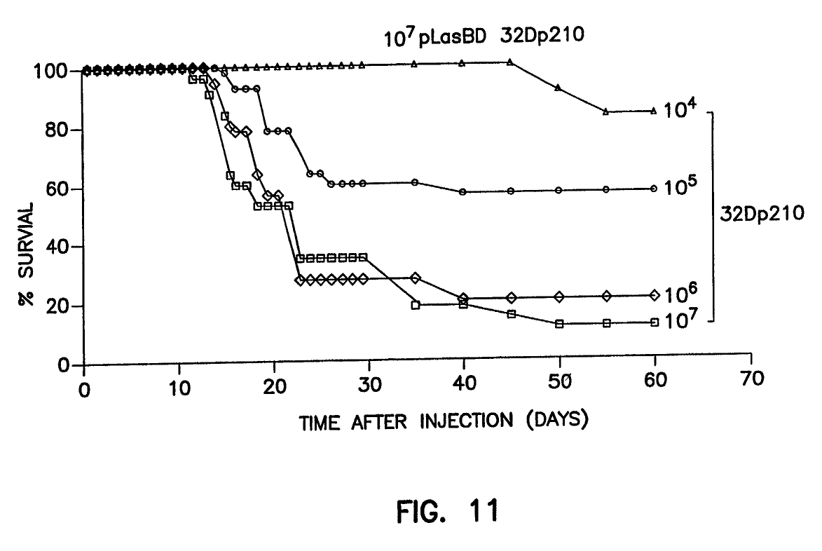

Approximately 10⁴-10⁷ 32D^{B/A} cells were transplanted IV into C3H animals. Animals were observed until day 100 after injection. Seventy percent of animals receiving 10⁶ or 10⁷ untransduced 32D^{B/A} cells and 50% of animals receiving 10⁵ untransduced 32D^{B/A} cells succumbed of leukemic infiltration by day 25 post-infusion. By day 60, 20% of animals receiving 10⁴, 55% of animals receiving 10⁵, and 80% of animals receiving 10⁶ and 95% of animals receiving 10⁷ untransduced cells died (FIG. 11). The majority of animals receiving 10⁶ and 10⁷ 32D^{B/A} cells succumbed between day 20 and 30. In contrast, 100% of the animals transduced with 10⁴-10⁷ bulk selected, or subcloned, LasBD cells survived more than 75 days post-transfusion. Thus, a double copy of a p210BCR/ABL ASO eliminates tumorigenicity of 32D^{P210} cells by at least 4 logs in vivo.

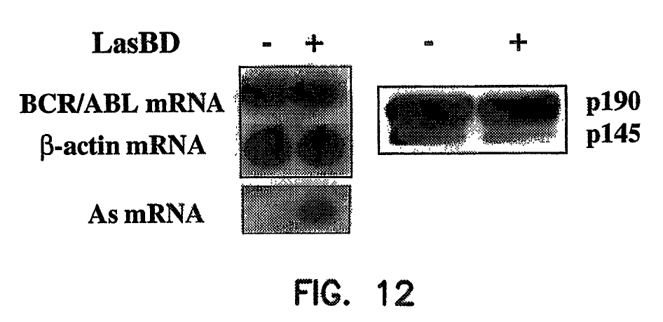

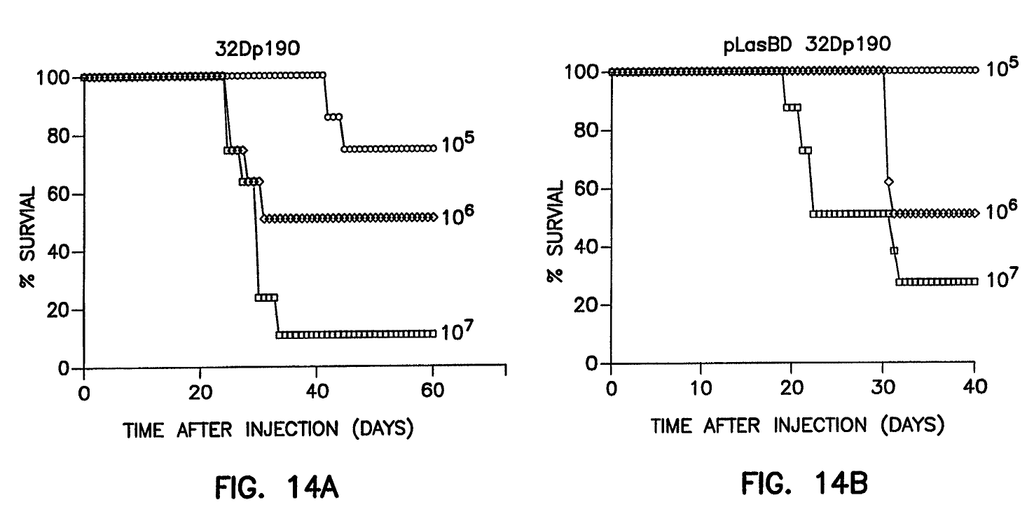

In order to determine the specificity of the LasBD effect, 32D^{P190} cells transduced with LasBD and selected on 0.25M MTX and IL3 for 14 days were examined for the levels of BCR/ABL mRNA and protein, survival ex vivo in the absence of IL3 and tumorigenicity in vivo. The transduction of LasBD did not affect p190BCR/ABL mRNA or protein levels (FIG. 12). Moreover, LasBD transduced 32D^{P190} cells, in contrast to LasBD transduced 32D^{P210} cells, were not IL3 dependent (FIG. 13). Furthermore, transfusion of LasBD transduced 32D^{P190} cells induced death at the same rate as when animals received untransduced 32D^{P190} cells (FIG. 14). These results show that the effect of the p210BCR/ABL ASO is specific for that breakpoint.

Thus, the transfer of a vector containing a MTX resistance gene and a BCR/ABL ASO can render target cells non-leukemic, and is one approach to prevent relapse due to transfused and/or systemic leukemia after auto-BMT in CML.

### EXAMPLE 5

### Efficacy of LasBD Vector in Human Ph+ Cells

To prepare an inoculum suitable for human hematopoietic cell transduction, an ASO-containing retroviral expression cassette was packaged in both PA317 cells and PG13 cells. Both cell lines and retroviral supernatants from either cell line are subjected to tests to determine viral titer and to determine the presence of contaminants such as mycoplasma, recombinant replication competent virus, bacteria and fungus. Supernatants that are found to be negative for the presence of contaminants, and which have adequate viral titers, are suitable for ex vivo human use.

To prepare human HSC for transduction, PBSC are mobilized with cytoxan (4 gm/m2, 1 dose) followed by G-CSF (5 μg/kg) from day 5 until the end of the PBPC collections. A total dose of 10×10⁶ CD34⁺ cells/kg is collected (5×10⁶/kg for the transplant and 5×10⁶/kg as back-up stem cells). CD34⁺ cells are selected from the 5×10⁶ cells/kg transplant sample using Ceprate™ columns. These CD34⁺ enriched cells are transduced with the supernatants containing the ASO-containing retroviral expression cassette, e.g., LasBD, using autologous stromal feeders, protamine, IL3, IL6 and SCF. Three transductions over a 72 hour period are performed.

Cytoxan/TBI (total body irradiation) or cytoxan/busulphan is administered to the transplant patient. On day 0, the patient receives the transduced cells. The patient is treated with G-CSF (5 μg/kg) until ANC>2,500 for 3 days.

After hematological engraftment is established (ANC>2,000 off G-CSF, red cell transfusion independent and Hb>9.5; platelets>80,000 untransfused), the patient is treated with MIX. MTX is administered in a dose escalation fashion. For example,

Month 1: 5 mg/m2/day PO during week 1, no therapy week 2, 3, and 4

Month 2: 7.5 mg/m2/day PO during week 1, no therapy week 2, 3, and 4

Month 3: 10 mg/m2/day PO during week 1, no therapy week 2, 3, and 4

Month 4: 15 mg/m2/day PO during week 1, no therapy week 2, 3, and 4

Patients are monitored by WBC, Hb and platelet levels, as well as liver and kidney function tests, which are evaluated at each treatment on day 0, day 7 and day 14.

If platelet, RBC or WBC levels fall under 80,000, 9 or 2,000, respectively,

on days 0 or 14, the next dose is not escalated. If platelet, RBC or WBC levels fall under 50,000, 8 or 1,000, respectively, on days 0 or 14, counts are reevaluated on an every other day basis and MTX administration stopped. If significant liver or kidney toxicity is observed (>3 fold normal levels of liver enzymes, >2 fold elevation of creatinine or BUN), no further MTX is administered.

BM and blood samples are obtained on day 0 of each MIX dose to determine the cellularity, presence of Ph+ cells, presence of Ph+ BCR/ABL mRNA negative progenitors, fraction of MTX resistant CFU-GM and LTC-IC. The results of these tests can determine the efficacy of ASO vector expression at the cellular and molecular level.

The complete disclosure of all patents, patent documents, and publications cited herein are incorporated by reference, as if individually incorporated. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. An expression cassette comprising: (a) a first nucleic acid molecule encoding resistance of a host cell to a cytotoxic agent that is employed to treat neoplastic disease operably linked to a first promoter functional in the host cell, and (b) a second nucleic acid molecule oprably linked to a second promoter functional in the host cell, wherein the second nucleic acid molecule encodes a RNA molecule or a polypeptide, the expression of which decreases the expression of a RNA molecule or polypeptide that is present in a malignant cell and not present in a corresponding non-malignant cell.

2. The expression cassette of claim 1 wherein the RNA molecule that is present in the malignant cell encodes a growth promoting gene product.

3. The expression cassette of claim 1 wherein the first nucleic acid molecule encodes dihydrofolate reductase.

4. The expression cassette of claim 3 wherein the dihydrofolate reductase is TYR²²-DHFR.

5. The expression cassette of claim 1 wherein the cytotoxic agent is methotrexate.

6. The expression cassette of claim 1 wherein the cytotoxic agent is taxol.

7. The expression cassett of claim 1 wherein the fist nucleic acid molecule encodes resistance to more than one cytotoxdc agent.

8. The expression cassette of claim 7 wherein the first nucleic acid molecule comprises the multidrug resistance gene.

9. The expression cassette of claim 1 wherein the polypeptide that is present in the malignant cell is P210^{BCR/ABL}.

10. The expression cassette of claim 3 wherein the polypeptide that is present in the malignant cell is P190^{BCR/ABL}.

11. The expression cassette of claim 1 wherein the polypeptide that is present in the malignant cell is encoded by a PML/RAR gene translocation.

12. The expression cassette of claim 1 wherein the second nucleic acid molecule comprises an antisense oligonucleotide.

13. The expression cassette of claim 12 wherein the RNA encoded by the antisense oligonucleotide is complementary to a RNA molecule that is present in a malignant cell and not present in a correspondinag non-malignant cell.

14. The expression cassette of claim 1 wherein the second nucleic acid molecule encodes an ribozyme.

15. The expression cassette of claim 14 wherein the ribozyme specifically cleaves a RNA molecule which encodes P210^{BCR/ABL}.

16. The expression cassette of claim 14 wherein the ribozyme specifically cleaves a RNA molecule which encodes P190^{BCR/ABL}.

17. The expression cassette of claim 14 wherein the ribozyme specifically cleaves a RNA molecule which encodes a polypeptide produced by a PML/RAR gene translocation.

18. The expression cassette of claim 1 wherein the second nucleic acid molecule encodes an antibody fragment.

19. The expression cassette of claim 18 wherein the antibody fragment specifically binds P210^{BCR/ABL}.

20. The expression cassette of claim 18 wherein the antibody fragment specifically binds P190^{BCR/ABL}.

21. The expression cassette of claim 18 wherein the antibody fragment specifically binds a polypeptide encoded by a PML/RAR gene translocation.

22. A method of preparing a hematopoietic stem cell which is resistant to a cytotoxic agent comprising:
(a) introducing into a hematopoietic stem cell a preselected DNA molecule so as to provide a transduced hematopoietic stem cell, wherein said preselected DNA molecule comprises
(i) a first DNA segment comprising a gene encoding resistance of a host cell to a cytotoxic agent that is employed to treat neoplastic disease operably linked to a first promoter functional in the hematopoietic stem cell, and
(ii) a second DNA segment operably liked to second promoter functional in the hematopoietic stem cell, wherein the second DNA segment encodes a RNA molecule or a polypeptide, the expression of which decreases the expression of a RNA molecule or polypeptide that is present in a malignant cell and not present in a corresponding non-malignant cell; and
(b) expressing the first DNA segment in the transduced cell in an amount effective to confer resistance to the cytotoxic agent to the transduced cell and expressing the second DNA segment in the transduced cell in an amount effective to inhibit or decrease the level of the RNA molecule or polypeptide which is present in the malignant cell.

23. A method for rendering a host mammal resistant to a cytotoxic agent, comprising:
introducing a population of transduced hematopoietic cells into a mammalian host wherein the genome of the transduced cells is augmented with a preselected DNA molecule comprising (i) a first DNA segment comprising a gene which encodes resistance to a cytotoxic agent that is employed to treat neoplastic disease operably linked to a first promoter functional in the traduced cell, and (ii) a second DNA segment operably linked to a second promoter functional in the transduced cells, wherein the second DNA segment encodes a RNA or polypeptide, the expression of which decreases the expression of a RNA molecule or polypeptide that is present in a malignant cell and not present in a corresponding non-malignant cell, so that the transduced cells are maintained in the host and impart host resistance to the cytotoxic agent and the transduced cells do not express the RNA molecule or polypeptide which is present in a malignant cell.

24. The method of claim 22 further comprising introducing the transduced cell into a mammalian host.

25. The method of claim 22 or 23 wherein the first DNA segment encodes dihydrofolate reductase.

26. The method of claim 22 or 23 wherein the first DNA segment comprises the multi-drug resistance gene.

27. The method of claim 25 wherein the dihydrofolate reductase is TYR²²-DHFR.

28. The method claim 22 or 23 wherein the cytotoxic agent is methotrexate.

29. The method of claim 22 or 23 wherein the cytotoxic agent is taxol.

30. The method of claim 22 or 23 wherein the polypeptide that is present in the malignant cell is P210^{BCR/ABL}.

31. The method of claim 22 or 23 wherein the polypeptide that is present in the malignant cell is P190_{BCR/ABL}.

32. The method of claim 22 or 23 wherein the polypeptide that is present in the malignant cell is encoded by a PML/RAR gene translocation.

33. The method of claim 22 or 23 wherein the second DNA segment encodes an antisense oligonucleotide.

34. The method of claim 33 wherein the RNA encoded by the antisense oligonucleatide is complementary to a RNA molecule tha is present in a malignant cell and not present in a corresponding non-malignant cell.

35. The method of claim 22 or 23 wherein the second DNA segment encodes an ribozyme.

36. The method of claim 35 wherein the ribozyme specifically cleaves a RNA molecule which encodes P210^{BCR/ABL}.

37. The method of claim 35 wherein the ribozyme specificaly cleaves a RNA molecule which encodes P190^{BCR/ABL}.

38. The method of claim 35 wherein the ribozyme specifically cleaves a RNA molecule which encodes a polypeptide produced by a PML/RAR gene translocation.

39. The method of claim 22 or 23 wherein the second nacleic acid molecule encodes an antibody fragment.

40. The method of claim 39 wherein the antibody fragment specifically binds P210^{BCR/ABL}.

41. The method of claim 39 wherein the antibody fragment specifically binds P190^{BCR/ABL}.

42. The method of claim 39 wherein the antibody fragment specifically binds a polypeptide encoded by a PML/RAR gene translocation.

43. The method of claim 23 wherein the hematopoietic cells comprise Ph⁺ cells.

44. The method of claim 23 further comprising administering a therapeutically effective amount of the cytotoxic agent to the host.
